(19) United States
(12) Patent Application Publication
(10) Pub. No.: US 2026/0309286 A1
(43) Pub. Date: Oct. 8, 2026

(54) GAS ODORIZATION APPARATUS, CONTROL, AND ASSOCIATED METHODS

(71) Applicant: TPE Midstream LLC, Tulsa, OK (US)

(72) Inventor: Douglas A. Sahm, Lutz, FL (US)

(21) Appl. No.: 19/490,258

(22) PCT Filed: Jun. 21, 2024
(86) PCT No.: PCT/US2024/035058

§ 371 Date: Dec. 4, 2025

Related U.S. Application Data
(60) Provisional application No. 63/510,023, filed on Jun. 23, 2023.

(51) Int. Cl. F17D 5/02; F17D 1/04; F17D 3/10; F17D 3/18; F17D 5/00

(52) U.S. Cl. CPC F17D 5/02; F17D 1/04; F17D 3/10; F17D 3/18; F17D 5/005

(57) ABSTRACT

Example gas odorization apparatus, control, and associated methods are disclosed herein. An example apparatus includes a pipe to contain a gas, the gas having a first concentration of odorant, and a fluid evacuation system coupled to the pipe and to a second location, the fluid evacuation system to evacuate the gas from the pipe and pump the gas to the second location, the gas at the second location having a second concentration of odorant greater than the first concentration of odorant.

### RELATED APPLICATION

This patent claims priority to U.S. Provisional Application No. 63/510,023, titled “Gas Odorization Apparatus, Control, and Associated Methods,” filed Jun. 23, 2023. U.S. Provisional Application No. 63/510,023 is hereby incorporated by reference in its entirety.

### FIELD OF THE DISCLOSURE

This disclosure relates generally to natural gas pipelines and, more particularly, to gas odorization apparatus, control, and associated methods.

### BACKGROUND

Pipelines are used to transport natural gas between two or more locations. Natural gas can include methane and/or one or more other constituent gases that are odorless and colorless. Thus, leakage of natural gas from a pipeline can be difficult to detect. Typically, an odorant (e.g., mercaptan, tetrahydrothiophene (THT), etc.) is injected into a gas stream to odorize the natural gas, thereby enabling detection of the odorized natural gas in case of leakage.

### BRIEF DESCRIPTION OF THE DRAWINGS

FIG. 1 illustrates an example environment including an example fluid evacuation system operatively coupled to an example pipe to implement an example gas odorization procedure in accordance with teachings of this disclosure.

FIG. 2A illustrates the example fluid evacuation system and the example pipe of FIG. 1, where the fluid evacuation system is fluidly coupled to an example gas storage.

FIG. 2B illustrates the example fluid evacuation system and the example pipe of FIGS. 1 and/or 2A, where the fluid evacuation system is fluidly coupled to an upstream pipe.

FIG. 3A is a schematic illustration of the example fluid evacuation system of FIGS. 1, 2A, and/or 2B.

FIG. 3B illustrates example compressor units of FIG. 3A configured for electrical actuation.

FIG. 3C illustrates a perspective view of an example linear actuator of FIG. 3B.

FIG. 4 is a flowchart representative of example operations to implement an example gas odorization procedure as described in connection with FIG. 1.

FIG. 5 is a flowchart representative of example operations to implement an example gas redistribution procedure as described in connection with FIGS. 2A and/or 2B.

FIG. 6 is a flowchart representative of example machine readable instructions and/or example operations that may be executed, instantiated, and/or performed by example programmable circuitry to implement the control system circuitry of FIG. 1.

FIG. 7 is a block diagram of an example processing platform including programmable circuitry structured to execute, instantiate, and/or perform the example machine readable instructions and/or perform the example operations of FIG. 6 to implement the control system circuitry of FIG. 1.

In general, the same reference numbers will be used throughout the drawing(s) and accompanying written description to refer to the same or like parts. The figures are not necessarily to scale. Instead, the thickness of the layers or regions may be enlarged in the drawings. Although the figures show layers and regions with clean lines and boundaries, some or all of these lines and/or boundaries may be idealized. In reality, the boundaries and/or lines may be unobservable, blended, and/or irregular.

As used herein, unless otherwise stated, the term “above” describes the relationship of two parts relative to Earth. A first part is above a second part, if the second part has at least one part between Earth and the first part. Likewise, as used herein, a first part is “below” a second part when the first part is closer to the Earth than the second part. As noted above, a first part can be above or below a second part with one or more of: other parts therebetween, without other parts therebetween, with the first and second parts touching, or without the first and second parts being in direct contact with one another.

As used in this patent, stating that any part (e.g., a layer, film, area, region, or plate) is in any way on (e.g., positioned on, located on, disposed on, or formed on, etc.) another part, indicates that the referenced part is either in contact with the other part, or that the referenced part is above the other part with one or more intermediate part(s) located therebetween.

As used herein, connection references (e.g., attached, coupled, connected, and joined) may include intermediate members between the elements referenced by the connection reference and/or relative movement between those elements unless otherwise indicated. As such, connection references do not necessarily infer that two elements are directly connected and/or in fixed relation to each other. As used herein, stating that any part is in “contact” with another part is defined to mean that there is no intermediate part between the two parts.

Unless specifically stated otherwise, descriptors such as “first,” “second,” “third,” etc., are used herein without imputing or otherwise indicating any meaning of priority, physical order, arrangement in a list, and/or ordering in any way, but are merely used as labels and/or arbitrary names to distinguish elements for ease of understanding the disclosed examples. In some examples, the descriptor “first” may be used to refer to an element in the detailed description, while the same element may be referred to in a claim with a different descriptor such as “second” or “third.” In such instances, it should be understood that such descriptors are used merely for identifying those elements distinctly within the context of the discussion (e.g., within a claim) in which the elements might, for example, otherwise share a same name.

As used herein, “approximately” and “about” modify their subjects/values to recognize the potential presence of variations that occur in real world applications. For example, “approximately” and “about” may modify dimensions that may not be exact due to manufacturing tolerances and/or other real world imperfections as will be understood by persons of ordinary skill in the art. For example, “approximately” and “about” may indicate such dimensions may be within a tolerance range of +/−10% unless otherwise specified in the below description.

### DETAILED DESCRIPTION

Buildings, plants, factories, and other facilities commonly use natural gas for various purposes such as heating, power generation, transportation, etc. Pipelines are used to transport natural gas between two or more locations. In some cases, leakage of natural gas from a pipe may occur when the pipe is ruptured and/or otherwise damaged (e.g., when the pipe is unintentionally struck during construction on and/or near the pipe). Natural gas is composed of methane and/or one or more other constituent gases (e.g., carbon dioxide, water vapor, ethane, propane, etc.) that, when leaked to the environment, can be harmful when inhaled and/or may pose a safety concern due to a risk of accidental combustion. Moreover, natural gas is naturally odorless and colorless, such that leakage of natural gas may be difficult to detect with human senses alone.

Typically, natural gas is odorized to facilitate detection of the natural gas in the event of leakage. For instance, an odorizer (e.g., an odorizing system) is used to provide odorant to a gas prior to commissioning of a pipeline. In some cases, the odorant can include mercaptan (e.g., tertiary butyl mercaptan, methyl mercaptan, ethyl mercaptan, etc.) and/or other chemicals (e.g., tetrahydrothiophene (THT), dimethyl sulfide, etc.). In some cases, one or more different types of odorizers may be used. For instance, a wick-type odorizer utilizes a wick that is saturated with odorant and is introduced into a gas stream to odorize the gas moving across the wick. Alternatively, an injection-type odorizer can inject small amounts of odorant into a pipe to odorize the gas therein.

In some instances, odor of the gas fades over time (e.g., due to absorption of the odorant by the surrounding pipe). Accordingly, the gas may periodically necessitate re-odorization by introduction of additional odorant to the pipe. Commonly, a pickling procedure is used to re-odorize the gas in the pipe. In a known pickling procedure, the gas in the pipe is saturated with a relatively large amount of odorant, and flow of the gas through the pipe is resumed after a period of time to enable distribution of the odorant throughout a remainder of the pipe. In some instances, when such large amounts of odorant are used, the odor can be detected at large distances (e.g., miles) away from the pipe, resulting in unnecessary concern and/or false alarms from residents in a surrounding area.

Examples disclosed herein evacuate unodorized (and/or under odorized) gas from a pipe for re-odorization, storage, and/or transmission to another location. An example fluid evacuation system disclosed herein is operatively coupled to an example pipe and, upon activation, the fluid evacuation system evacuates unodorized (and/or under odorized) gas from the pipe. In some examples, an example odorizer is fluidly coupled to the fluid evacuation system to receive the evacuated gas therefrom, and the odorizer is further coupled to the pipe. In such examples, the odorizer can be a wick-type odorizer, an injection-type odorizer, or another type of odorizer. In some examples, the odorizer provides odorant to the gas from the fluid evacuation system, and the re-odorized gas is returned to the pipe. Additionally or alternatively, the gas from the fluid evacuation system can be provided to an example gas storage for storage and/or transportation. In some such examples, unodorized gas in the gas storage can be provided to one or more second locations (e.g., a second pipe and/or a location upstream and/or downstream of the pipe), such that the unodorized gas from the gas storage mixes with odorized gas at the second location. In some examples, the gas storage can be omitted, such that the gas from the fluid evacuation system can be directly routed to the second location.

Advantageously, examples disclosed herein enable unodorized gas to be evacuated from a pipe, re-odorized, and returned to the pipe, thus reducing risk of harm associated with undetected leakage of unodorized gas from the pipe. Further, examples disclosed herein re-odorize the gas by cycling the gas through an odorizer fluidly coupled to a fluid evacuation system, thus eliminating the need for oversaturating a pipe with odorant using a known pickling procedure. Accordingly, examples disclosed herein reduce a likelihood of detection of the odorant from large distances away from the pipe, thus reducing unnecessary concern and/or false alarms from surrounding residents.

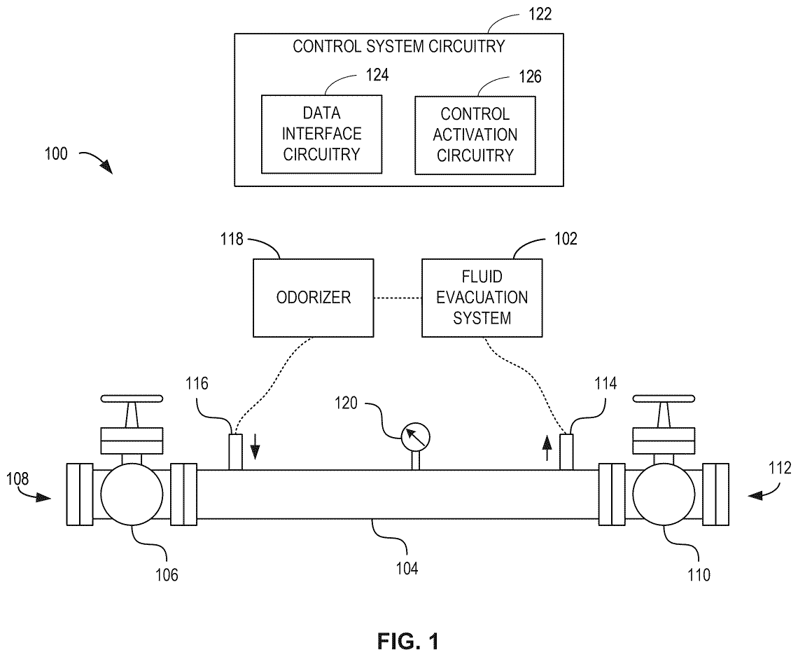

FIG. 1 illustrates an example environment 100 including an example fluid evacuation system 102 operatively coupled to an example pipe 104 to implement an example gas odorization procedure in accordance with teachings of this disclosure. In the illustrated example of FIG. 1, the pipe 104 includes a first example valve (e.g., ball valve) 106 at a first example end 108 of the pipe 104 and a second example valve 110 at a second example end 112 of the pipe 104. In some examples, prior to performance of a gas odorization procedure, the pipe 104 is sealed at one or both ends by closing the first valve 106 and/or the second valve 110.

In the example of FIG. 1, the pipe 104 includes a first example port 114 and a second example port 116 through which gas can flow into and/or out of the pipe 104. In this example, the fluid evacuation system 102 is fluidly and/or operatively coupled to the pipe 104 at the first port 114. In some examples, an example odorizer (e.g., an odorizing system) 118 is fluidly coupled to the fluid evacuation system 102 and to the pipe 104 at the second port 116. In some examples, the odorizer 118 is at least one of a wick-type odorizer or an injection-type odorizer. In some examples, a different type of odorizer (e.g., a bypass odorizer, a pulse bypass odorizer, a drip odorizer, etc.) may be used instead. In some examples, the fluid evacuation system 102 and the odorizer 118 are used to perform the example gas odorization procedure on the example pipe 104.

In some examples, the gas odorization procedure is performed when gas in the pipe 104 is unodorized and/or under odorized. For example, the gas may become unodorized and/or under odorized when the gas has remained stationary (e.g., not moving) in the pipe for an extended duration (e.g., one year, two years, etc.). To begin the gas odorization procedure, the fluid evacuation system 102 is turned on (e.g., activated or triggered by an operator and/or by a control system operatively coupled to the fluid evacuation system 102) to begin evacuating and/or compressing gas from the pipe 104. In this example, the fluid evacuation system 102 directs the evacuated gas from the pipe 104 to and/or through the odorizer 118. In some examples, as the gas passes through and/or across the odorizer 118, the odorizer 118 provides odorant to re-odorize the gas. In some examples, after re-odorization of the gas by the odorizer 118, the fluid evacuation system 102 causes the re-odorized gas to return to the pipe 104 via the second port 116. In some examples, the fluid evacuation system 102 continues to evacuate the gas from the pipe 104 and/or cycle the gas through the odorizer 118 until a threshold amount of the gas has been re-odorized.

In the illustrated example of FIG. 1, an example meter (e.g., a sensor, a gauge) 120 is operatively coupled to the pipe 104 to measure and/or display one or more characteristics of the gas therein. For example, the meter 120 can measure a pressure of the gas in the pipe 104, a volume of the gas in the pipe 104, a concentration of odorant relative to the gas in the pipe 104, etc. In some examples, the meter 120 provides measurement data to example control system circuitry 122 for storage and/or for use in controlling operation of the fluid evacuation system 102 and/or the odorizer 118. In some examples, the fluid evacuation system 102 evacuates the gas from the pipe 104 based on the one or more characteristics measured and/or output by the meter 120. For example, a gas evacuation procedure by the fluid evacuation system 102 may be initiated (e.g., by the control system circuitry 122 and/or by an operator) when the concentration of odorant does not satisfy (e.g., is less than) a threshold concentration, and the fluid evacuation system 102 can continue evacuation of the gas while the pressure of the gas in the pipe 104 satisfies (e.g., is greater than or equal to) a threshold pressure and/or when the volume of the gas evacuated from the pipe 104 satisfies (e.g., is greater than or equal to) a threshold volume.

In the example of FIG. 1, the control system circuitry 122 controls the fluid evacuation system 102 and/or the odorizer 118 to implement an example gas odorization procedure. In some examples, the control system circuitry 122 can be omitted, and an operator can manually control the operation of the fluid evacuation system 102 and/or the odorizer 118. In the example of FIG. 1, the control system circuitry 122 includes example data interface circuitry 124 and example control activation circuitry 126. The example data interface circuitry 124 is communicatively coupled to the meter 120 to obtain and/or access measurement data therefrom. For example, the data interface circuitry 124 can determine, based on the measurement data, the characteristic(s) of the gas in the pipe 104 including at least one of the pressure of the gas, the volume of the gas, the concentration of odorant, etc.

In the illustrated example of FIG. 1, the control activation circuitry 126 is operatively and/or communicatively coupled to the fluid evacuation system 102 and/or the odorizer 118. In some examples, the control activation circuitry 126 determines whether to cause operation of the fluid evacuation system 102 and/or the odorizer 118 based on the characteristic(s) of the gas determined and/or obtained by the data interface circuitry 124. For example, the control activation circuitry 126 determines whether to initiate the gas odorization procedure based on whether the characteristic(s) satisfy one or more thresholds. In some examples, the control activation circuitry 126 initiates the gas odorization procedure in response to determining that the concentration of odorant in the pipe 104 does not satisfy (e.g., is less than) a threshold concentration. In some examples, one or more different thresholds can be used to initiate the gas odorization procedure.

In some examples, when the control activation circuitry 126 determines that the gas odorization procedure is to be performed, the control activation circuitry 126 activates (e.g., turns on) the fluid evacuation system 102 to direct the gas from the pipe 104 to and/or through the odorizer 118. In some examples, the control activation circuitry 126 adjusts one or more operational parameters (e.g., a compression rate, a total compression, etc.) of the fluid evacuation system 102 based on the gas characteristic(s) (e.g., the pressure of the gas and/or the volume of the gas in the pipe 104). Additionally or alternatively, the control activation circuitry 126 can adjust, based on the gas characteristic(s), one or more operational parameters of the odorizer 118, such as an injection rate of odorant (e.g., when the odorizer 118 is an injection-type odorizer) and/or an amount of the odorant provided to the gas.

In some examples, the control activation circuitry 126 deactivates (e.g., shuts off) the fluid evacuation system 102 and/or the odorizer 118 in response to determining that the gas odorization procedure is complete. For example, the control activation circuitry 126 deactivates the fluid evacuation system 102 and/or the odorizer 118 in response to determining that the concentration of odorant in the pipe 104 satisfies (e.g., is greater than) a threshold concentration, the pressure of the gas in the pipe 104 satisfies a threshold pressure, a duration for which the fluid evacuation system 102 is operating satisfies a threshold duration, etc.

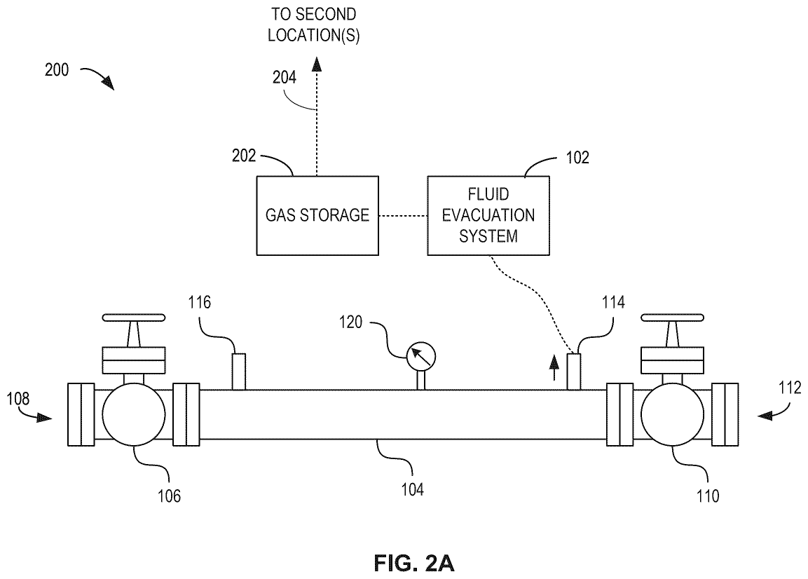

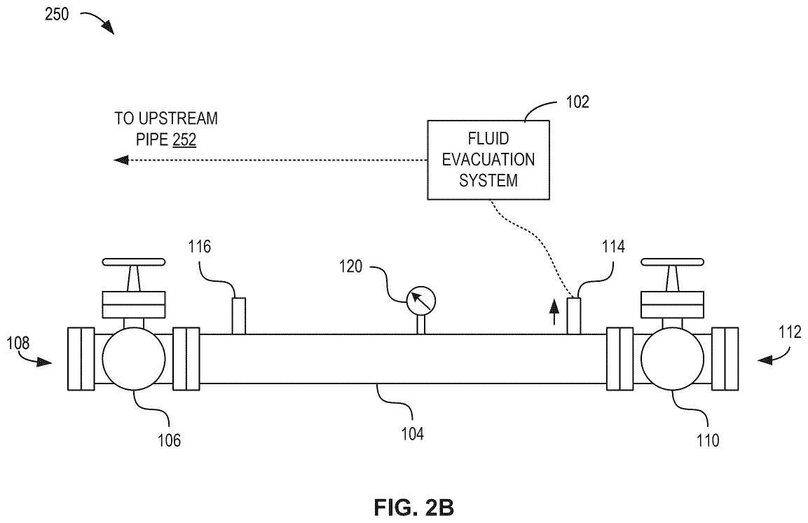

FIG. 2A illustrates a second example environment 200 including the example fluid evacuation system 102 implemented on the example pipe 104 of FIG. 1, where the fluid evacuation system 102 is fluidly coupled to an example gas storage 202. In the example of FIG. 2A, an operator and/or the control system circuitry 122 of FIG. 1 determines that a gas redistribution procedure is to be initiated when the concentration of odorant relative to the gas in the pipe 104 (e.g., as indicated by the example meter 120) does not satisfy (e.g., is less than) a threshold concentration. In some examples, the operator and/or the control system circuitry 122 of FIG. 1 activates the fluid evacuation system 102 to evacuate gas (e.g., unodorized and/or under odorized gas) from the pipe 104. In this example, the gas is directed from the fluid evacuation system 102 to the example gas storage 202 for storage therein. In some examples, the fluid evacuation system 102 continues to compress and/or provide the gas to the gas storage 202 until the concentration of odorant satisfies the threshold concentration and/or an amount of gas in the gas storage 202 satisfies a threshold amount. In some examples, the operator and/or the control system circuitry 122 deactivates (e.g., shuts off) the fluid evacuation system 102 in response to determining that the concentration of odorant satisfies the threshold concentration and/or the amount of gas in the gas storage 202 satisfies the threshold amount.

In some examples, the gas storage 202 can be disconnected from the fluid evacuation system 102 after deactivation of the fluid evacuation system 102, and the gas from the gas storage 202 can be provided to one or more second example locations 204. For example, the second location(s) 204 can include one or more second pipes containing odorized gas, and the gas storage 202 can be fluidly coupled to the one or more second pipes such that the unodorized and/or under odorized gas from the gas storage 202 can be provided to the second pipe(s) to mix with the odorized gas therein. In some such examples, the resultant combination of gases is sufficiently odorized (e.g., has a concentration of odorant at or above a threshold concentration) such that re-odorization of the gas is not necessary. In some examples, the second location(s) 204 can include a location upstream and/or downstream of the pipe 104. In some examples, the gas storage 202 can be omitted, and the fluid evacuation system 102 is fluidly coupled to the second pipe(s) to pump unodorized and/or under odorized gas from the pipe 104 to the second pipe(s). In some examples, at least one odorizer (e.g., the odorizer 118 of FIG. 1) can be fluidly coupled between the fluid evacuation system 102 and the gas storage 202 to odorize the gas provided to the gas storage 202. Additionally or alternatively, the at least one odorizer can be fluidly coupled between the gas storage 202 and the second location(s) 204 to odorize the gas flowing to the second location(s) 204. In some examples, odorized gas can be introduced into the pipe 104 after evacuation of the unodorized and/or under odorized gas therefrom.

FIG. 2B illustrates a third example environment 250 including the example fluid evacuation system 102 implemented on the example pipe 104 of FIGS. 1 and/or 2A, where the fluid evacuation system 102 is fluidly coupled to an example upstream pipe 252. In the example of FIG. 2B, the gas storage 202 of FIG. 2A is omitted, and the fluid evacuation system 102 is coupled to the pipe 104 and to the upstream pipe 252. In some examples, the upstream pipe 252 corresponds to one or more second pipes upstream of the pipe 104 and/or fluidly coupled to the first end 108 of the pipe 104, where the upstream pipe 252 can contain odorized gas (e.g., a second gas having a concentration of odorant at or above a threshold concentration) in some examples. In the example of FIG. 2B, the operator and/or the control system circuitry 122 of FIG. 1 can activate the fluid evacuation system 102 to evacuate gas (e.g., unodorized and/or under odorized gas) from the pipe 104. In this example, the fluid evacuation system 102 directs and/or provides the unodorized (or under-odorized) gas from the pipe 104 to the upstream pipe 252 to mix with odorized gas therein. In some such examples, the resultant combination of gases is sufficiently odorized (e.g., has a concentration of odorant at or above the threshold concentration) such that re-odorization of the gas is not necessary. Stated differently, while the unodorized and/or under-odorized gas from the pipe 104 corresponds to a first concentration of odorant (e.g., less than the threshold concentration), the resultant combination of gases in the upstream pipe 252 corresponds to a second concentration of odorant (e.g., greater than or equal to the threshold concentration). In some examples, the odorizer 118 of FIG. 1 can be fluidly coupled between the fluid evacuation system 102 and the upstream pipe 252 to provide odorant to the gas prior to the gas being provided (e.g., pumped) to the upstream pipe 252. In some examples, new (e.g., odorized) gas can be introduced into the pipe 104 (e.g., from the upstream pipe 252, from a separate pipe, from a gas storage, etc.) after evacuation of the unodorized and/or under-odorized gas from the pipe 104.

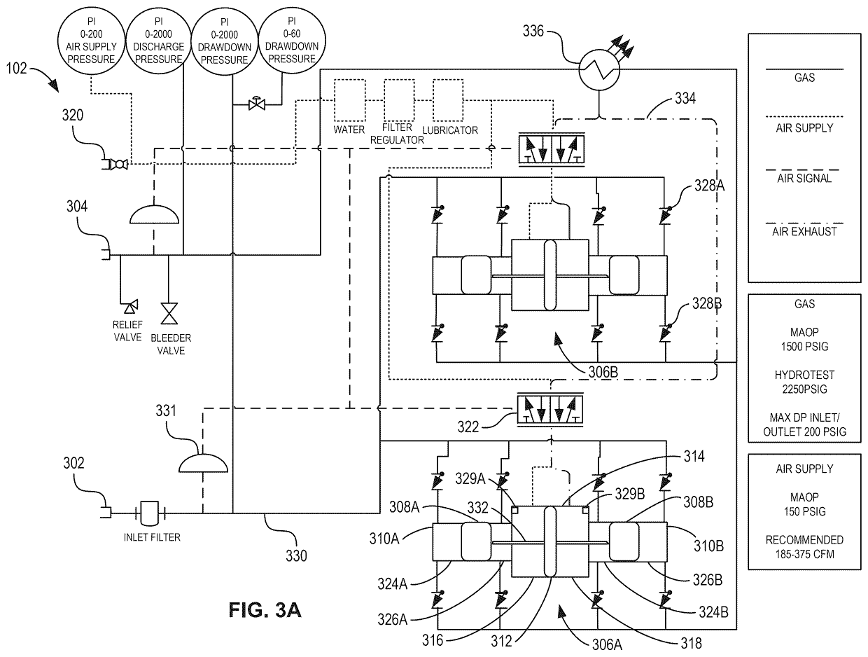

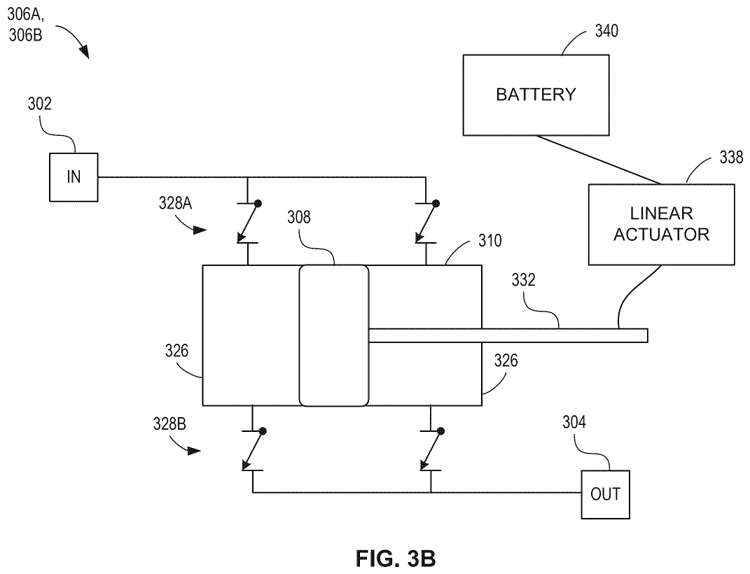

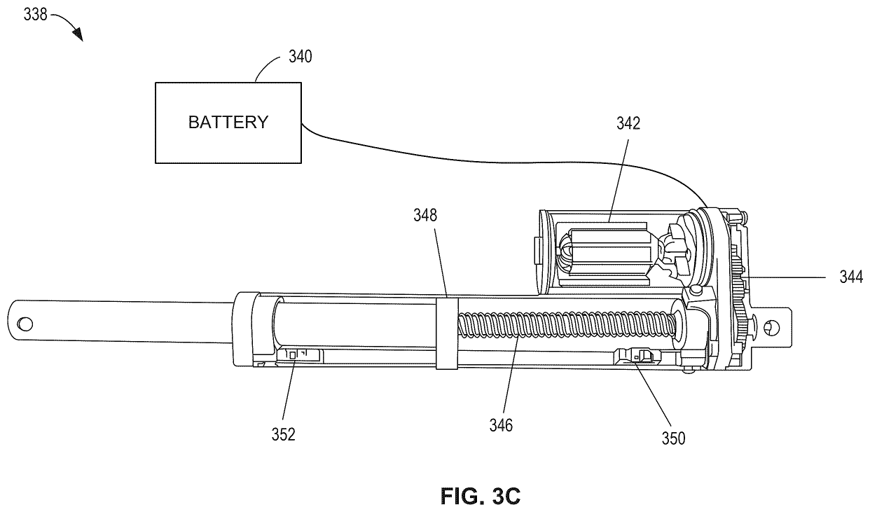

FIG. 3A is a schematic illustration of the example fluid evacuation system 102 of FIGS. 1, 2A, and/or 2B. The example fluid evacuation system 102 is configured to transport fluid (e.g., gas) from a first location (e.g., the pipe 104 of FIGS. 1, 2A, and/or 2B) to a second location (e.g., the example odorizer 118 of FIG. 1, the example gas storage 202 of FIG. 2A, the second location(s) 204 of FIG. 2A, the upstream pipe 252 of FIG. 2B, a second pipe, etc.). The example fluid evacuation system 102 includes an example fluid intake 302 couplable to the pipe 104 and an example fluid discharge 304 couplable to the second location. Fluid is compressed by example compressor units 306A, 306B as the fluid flows from the fluid intake 302 to the fluid discharge 304. The compressor units 306A, 306B each include example compression pistons 308A, 308B implemented in example compression cylinders 310A, 310B, and an example air piston 312 implemented in an example air cylinder 314. The air cylinder 314 includes an example first chamber 316 and an example second chamber 318 coupled to an example air supply 320 via an example air control valve 322. The compression cylinders 310A, 310B include example third chambers 324A, 324B and example fourth chambers 326A, 326B coupled to the fluid intake 302 via inlet check valves 328A, and coupled to the fluid outlet via outlet check valves 328B.

In the illustrated example of FIG. 3A, fluid enters via the fluid intake 302 and flows to the compressor units 306A, 306B via example piping 330. The fluid enters the third chambers 324A, 324B and the fourth chambers 326A, 326B through the inlet check valves 328A. The inlet check valves 328A allow the fluid to flow unidirectionally from the fluid intake 302 to the compressor units 306A, 306B. The air control valve 322 also directs compressed air from the air supply 320 to enter the air cylinder 314. The air control valve 322 can alternate flow of the compressed air between the first chamber 316 and the second chamber 318. In the illustrated example of FIG. 3A, the air control valve 322 directs compressed air into the first chamber 316 in response to a first switch 329A being engaged, and directs compressed air into the second chamber 318 in response to a second switch 329B being engaged, where the first switch 329A and the second switch 329B are operatively coupled to the air control valve 322. In other examples, the air control valve 322 can switch a direction of flow of the compressed air based on a command and/or a signal from a computer and/or other processor communicatively coupled to the air control valve 322.

In the illustrated example of FIG. 3A, an under-pressure cutoff 331 is coupled to the piping 330 between the fluid intake 302 and the air control valve 322. In some examples, the under-pressure cutoff 331 can detect whether a pressure of the fluid in the piping 330 drops below a threshold pressure (e.g., 0 psi, 5 psi, etc.). In response to the under-pressure cutoff 331 determining that the pressure of the fluid has dropped below the cutoff pressure, the under-pressure cutoff 331 can send an air signal to the air control valve 322 to shut off the flow of compressed air into the compressor units 306A, 306B and, as such, prevent the compressor units 306A, 306B from further compressing the fluid.

In the illustrated example of FIG. 3A, in response to the air control valve 322 directing the compressed air to flow into the first chamber 316, the compressed air generates pressure on the air piston 312 to move the air piston 312 to the right (e.g., towards the second compression cylinder 310B). The air piston 312 is operatively coupled to the compression pistons 308A, 308B via an example rod 332, such that the compression pistons 308A, 308B move with the air piston 312. In response to the air piston 312 moving to the right and, thus, the compression pistons 308A, 308B moving to the right, the fluid in the fourth chambers 326A, 326B is compressed by the compression pistons 308A, 308B. Compressed fluid is expelled from the fourth chambers 326A, 326B and flows through the respective outlet check valves 328B towards the fluid discharge 304. The outlet check valves 328B allow the fluid to flow unidirectionally from the fluid intake 302 to the compressor units 306A, 306B.

In response to the air piston 312 being positioned to the right (in reference to the arrangement of FIG. 3A), the air piston 312 engages the second switch 329B coupled to the right side of the air cylinder 314. In response to the second switch 329B being engaged, the air control valve 322 stops the flow of compressed air to the first chamber 316 and directs the flow of compressed air to enter the second chamber 318. The compressed air from the first chamber 316 can be expelled to the atmosphere via air exhaust tubing 334. In some examples, the compressed air from the first chamber 316 can be used to cool the compressed fluid via an example heat exchanger 336 prior to the compressed air being expelled to the atmosphere.

In response to the air control valve 322 directing the flow of compressed air to enter the second chamber 318, the compressed air causes the air piston 312 and the compression pistons 308A, 308B to move to the left (e.g., toward the first compression cylinder 310A). The fluid in the third chambers 324A, 324B is compressed by the compression pistons 308A, 308B. The compressed fluid is expelled from the third chambers 324A, 324B and flows through the respective outlet check valves 328B towards the fluid discharge 304.

In response to the air piston 312 being positioned to the left (in reference to the arrangement of FIG. 3A), the air piston 312 engages the first switch 329A coupled to the left side of the air cylinder 314. In response to the first switch 329A being engaged, the air control valve 322 stops the flow of compressed air to the second chamber 318 and once again directs the flow of compressed air to enter the first chamber 316. In the illustrated example of FIG. 3A, the air control valve 322 continuously redirects the flow of compressed air between the first chamber 316 and the second chamber 318 to compress fluid entering the third chambers 324A, 324B and the fourth chambers 326A, 326B.

In the illustrated example of FIG. 3A, the fluid evacuation system 102 includes two compressor units (e.g., the first compressor unit 306A and the second compressor unit 306B). In other examples, to reduce a size of the fluid evacuation system 102, only one of the compressor units (e.g., the first compressor unit 306A or the second compressor unit 306B) is used. In other examples, multiple ones (e.g., three or more) of the compressor units are used. In such examples, the rate of compression and/or the differential pressure of the gas compressed by the fluid evacuation system 102 can be modified by selectively configuring an arrangement of the compressor units (e.g., in a series arrangement and/or in a parallel arrangement).

FIG. 3B illustrates the compressor units 306A, 306B of FIG. 3A configured for electrical, rather than pneumatic, actuation. In such examples, gas from the fluid intake 302 of FIG. 3A is not compressed using compressed air from the air supply 320, but rather is compressed via an example linear actuator 338. As such, in this example, the fluid evacuation system 102 does not include the air control valve 322, the air supply 320, and/or the air exhaust tubing 334 of FIG. 3A. The linear actuator 338 is coupled to and/or powered by an example battery 340.

In the illustrated example of FIG. 3B, the linear actuator 338 is operatively coupled to the rod 332 to move the gas piston 308 (e.g., the first compression piston 308A or the second compression piston 308B of FIG. 3A) inside the compression cylinder 310 (e.g., the first compression cylinder 310A or the second compression cylinder 310B of FIG. 3A). In this example, the linear actuator 338 is configured such that the gas piston 308 moves to the left when the linear actuator 338 extends, and the gas piston 308 moves to the right when the linear actuator 338 contracts. Alternatively, in other examples, the linear actuator 338 is configured such that the gas piston 308 moves to the left when the linear actuator 338 contracts, and the gas piston 308 moves to the right when the linear actuator 338 extends.

In this example, each of the compressor units 306A, 306B includes a single one of the compression pistons 308A, 308B and a corresponding one of the compression cylinders 310A, 310B. In such examples, each of the compressor units 306A, 306B includes a corresponding linear actuator 338. In other examples, the linear actuator 338 can be coupled to both of the compressor units 306A, 306B to operate the compressor units 306A, 306B simultaneously. In other examples, the compressor units 306A, 306B can include both of the compression pistons 308A, 308B operated by the linear actuator 338.

In the illustrated example of FIG. 3B, in response to the linear actuator 338 moving the gas piston 308 to the right, the gas in the fourth chamber 326 is compressed by the gas piston 308. Compressed gas is expelled from the fourth chamber 326 and flows through the respective outlet check valves 328B towards the fluid discharge 304. Alternatively, in response to the linear actuator 338 moving the gas piston 308 to the left, the gas in the third chamber 324 is compressed by the gas piston 308. Compressed gas is expelled from the fourth chamber 326 and flows through the respective outlet check valves 328B towards the fluid discharge 304. In this example, the linear actuator 338 continuously moves between an extended position and a contracted position to compress gas entering the third chamber 324 and the fourth chamber 326 until the gas is evacuated from the first location (e.g., coupled to the fluid intake 302) and transferred to the second location (e.g., coupled to the fluid discharge 304).

FIG. 3C illustrates a perspective view of the example linear actuator 338 of FIG. 3B. The example linear actuator 338 includes an example motor 342 coupled to the battery 340 of FIG. 3B, an example gear box 344, an example lead screw 346, an example drill nut 348, an example retract limit switch 350, and an example extend limit switch 352. In the illustrated example of FIG. 3C, rotation of the motor 342 causes corresponding rotation of the lead screw 346 via the gear box 344. The rotation of the lead screw 346 causes linear travel of the drill nut 348 along the lead screw 346 and, as such, causes the linear actuator 338 to extend or retract based on a direction of rotation of the motor 342 and/or the lead screw 346. For example, the linear actuator 338 extends in response to the motor 342 rotating in a first direction, and the linear actuator 338 retracts in response to the motor 342 rotating in a second direction, where the second direction is opposite from the first direction.

In the illustrated example of FIG. 3C, in response to the linear actuator 338 being fully extended, the drill nut 348 engages the extend limit switch 352. In such examples, the extend limit switch 352 sends a first electrical signal to the motor 342. In some examples, the first electrical signal causes the motor 342 to stop rotating and/or reverse the direction of rotation (e.g., from the first direction to the second direction). Alternatively, in response to the linear actuator 338 being fully retracted, the drill nut 348 engages the retract limit switch 350. In such examples, the retract limit switch 350 sends a second electrical signal to the motor 342. In some examples, where the first electrical signal causes the motor 342 to stop rotating and/or reverse the direction of rotation (e.g., from the second direction to the first direction). As such, repeatedly engaging the retract limit switch 350 and the extend limit switch 352 causes linear reciprocal travel of the linear actuator 338 to compress the gas in the compression cylinder 310 of FIG. 3B.

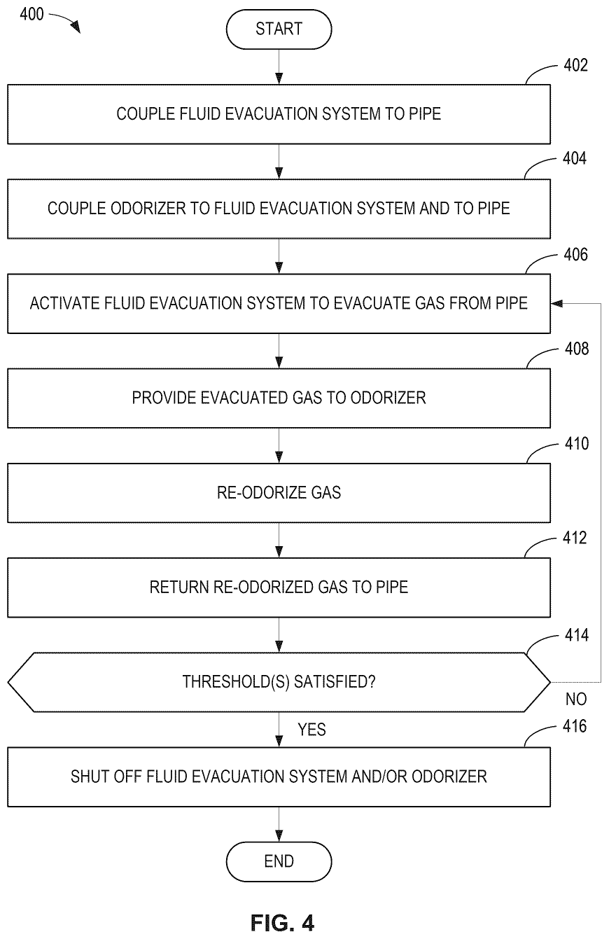

FIG. 4 is a flowchart representative of example operations 400 to implement an example gas odorization procedure as described in connection with FIG. 1. The example operations 400 begin with unodorized and/or under odorized gas in the example pipe 104 of FIGS. 1, 2A, and/or 2B. In some examples, the operations 400 begin when an operator determines, based on measurement data from the example meter 120 of FIGS. 1, 2A, and/or 2B, that a concentration of odorant in the pipe 104 is less than a threshold concentration. In some examples, the operations 400 begin when an operator determines that the pipe 104 has been out of operation (e.g., the gas and/or the odorant in the pipe 104 has remained idle) for at least a threshold duration.

At block 402, the example fluid evacuation system 102 is coupled to the example pipe 104. For example, the fluid evacuation system 102 is fluidly and/or operatively coupled to the pipe 104 at the first example port 114 of FIGS. 1, 2A, and/or 2B.

At block 404, the example odorizer 118 of FIG. 1 is coupled to the fluid evacuation system 102 and to the example pipe 104. For example, the odorizer 118 is fluidly and/or operatively coupled to the fluid evacuation system 102 to receive gas therefrom. In some examples, the odorizer 118 is further fluidly coupled to the pipe 104 at the second example port 116 of FIGS. 1, 2A, and/or 2B. Additionally or alternatively, the odorizer 118 can be coupled to the example gas storage 202 of FIG. 2A and/or to one or more second locations (e.g., a second pipe, a location upstream and/or downstream of the pipe 104, etc.).

At block 406, the example fluid evacuation system 102 is activated to evacuate gas (e.g., unodorized and/or under odorized gas) from the pipe 104. For example, an operator activates the fluid evacuation system 102 so that the fluid evacuation system 102 can begin compressing and/or evacuating gas from the pipe 104.

At block 408, the example fluid evacuation system 102 provides the evacuated gas to the example odorizer 118. For example, the fluid evacuation system 102 directs the gas from the pipe 104 to the odorizer 118 to enable flow of the gas through, across, and/or near the odorizer 118.

At block 410, the example odorizer 118 re-odorizes the gas provided to the odorizer 118 from the fluid evacuation system 102. For example, the odorizer 118 provides odorant to the gas provided to the odorizer 118. In some examples, the odorizer 118 is a wick-type odorizer including a wick saturated with odorant, where the gas is re-odorized as the gas flows across the wick. In some examples, the odorizer 118 is an injection-type odorizer that injects odorant into the gas as the gas flows through the odorizer 118. In some examples, one or more different types of odorizers (e.g., a bypass odorizer, a pulse bypass odorizer, a drip odorizer, etc.) can be used to provide the odorant to the gas.

At block 412, the re-odorized gas is returned to the pipe 104. For example, operation of the fluid evacuation system 102 causes the re-odorized gas to flow from the odorizer 118 to the pipe 104 via the second example port 116. Additionally or alternatively, in some examples, the fluid evacuation system 102 causes the re-odorized gas to flow to a second location (e.g., the example gas storage 202 of FIG. 2A, a second example pipe, etc.) fluidly coupled to the odorizer 118.

At block 414, an operator determines whether the re-odorized gas satisfies one or more example thresholds. For example, the operator monitors, based on the example meter 120 of FIGS. 1, 2A, and/or 2B, one or more parameters associated with the re-odorized gas. In some examples, the parameter(s) include an amount of gas that has been re-odorized, an amount (e.g., a concentration) of odorant in the pipe 104, a duration for which the fluid evacuation system 102 has been operating, etc. In some examples, in response to the operator determining that the re-odorized gas satisfies the example threshold(s) (e.g., block 414 returns a result of YES), control proceeds to block 416. Alternatively, in response to the operator determining that the re-odorized gas does not satisfy one or more of the threshold(s) (e.g., block 414 returns a result of NO), control returns to block 406.

At block 416, the fluid evacuation system 102 and/or the odorizer 118 is shut off and/or deactivated. For example, the operator shuts off (e.g., deactivates) the fluid evacuation system 102 so that gas is no longer being evacuated from the pipe 104 and/or provided to the odorizer 118. In some examples, the fluid evacuation system 102 and/or the odorizer 118 are removed (e.g., decoupled) from the pipe 104 upon completion of the gas odorization procedure.

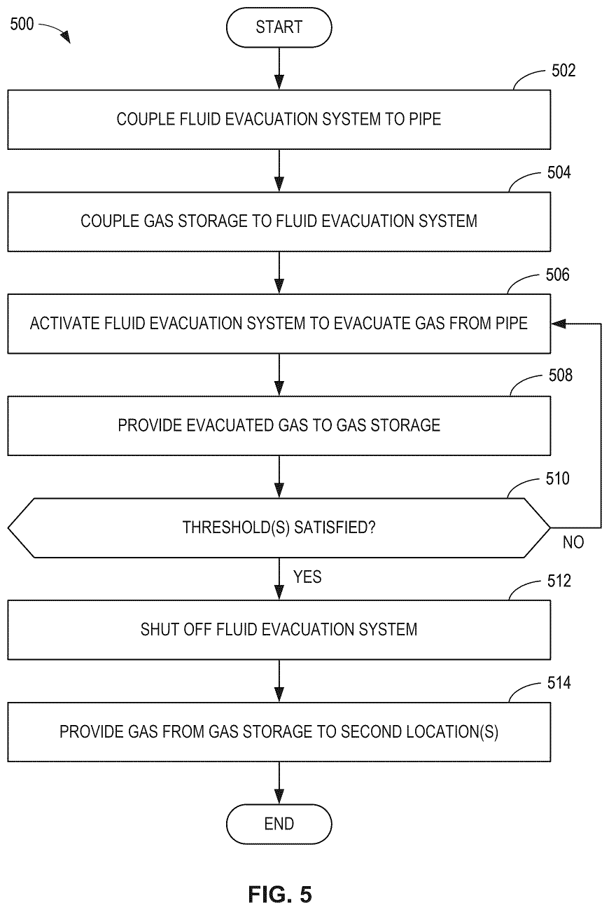

FIG. 5 is a flowchart representative of example operations 500 to implement an example gas redistribution procedure as described in connection with FIGS. 2A and/or 2B. The example operations 500 begin with unodorized and/or under odorized gas in the example pipe 104 of FIGS. 1, 2A, and/or 2B. In some examples, the operations 500 begin when an operator determines, based on measurement data from the example meter 120 of FIGS. 1, 2A, and/or 2B, that a concentration of odorant in the pipe 104 is less than a threshold concentration. In some examples, the operations 500 begin when an operator determines that the pipe 104 has been out of operation (e.g., the gas and/or the odorant in the pipe 104 has remained idle) for at least a threshold duration.

At block 502, the example fluid evacuation system 102 is coupled to the example pipe 104. For example, the fluid evacuation system 102 is fluidly and/or operatively coupled to the pipe 104 at the first example port 114 of FIGS. 1, 2A, and/or 2B.

At block 504, the example gas storage 202 of FIG. 2A is coupled to the example fluid evacuation system 102. For example, the gas storage 202 is fluidly and/or operatively coupled to the fluid evacuation system 102 to receive compressed gas therefrom. In some examples, the gas storage 202 may be omitted, and the fluid evacuation system 102 can be directly coupled to the second location(s) 204, to the upstream pipe 252 of FIG. 2B, etc.

At block 506, the example fluid evacuation system 102 is activated to evacuate gas (e.g., unodorized and/or under odorized gas) from the pipe 104. For example, an operator activates the fluid evacuation system 102 so that the fluid evacuation system 102 can begin compressing and/or evacuating the gas from the pipe 104.

At block 508, the example fluid evacuation system 102 provides the evacuated gas to the example gas storage 202. For example, the fluid evacuation system 102 compresses and/or provides the gas from the pipe 104 to the gas storage 202 for storage and/or transportation. Additionally or alternatively, when the gas storage 202 is omitted, the fluid evacuation system 102 provides the gas from the pipe 104 directly to the second location(s) 204, to the upstream pipe 252 of FIG. 2B, etc.

At block 510, an operator determines whether one or more example thresholds are satisfied. For example, the operator monitors, based on the example meter 120 of FIGS. 1, 2A, and/or 2B, whether a concentration of odorant in the pipe 104 satisfies a threshold concentration, a duration for which the fluid evacuation system 102 has been operating satisfies a threshold duration, an amount (e.g., a volume) of gas in the gas storage 202 satisfies a threshold amount (e.g., a threshold volume corresponding to a storage capacity of the gas storage 202), etc. In some examples, the threshold duration is based on an idle duration for which previously-odorized gas has remained idle in the pipe 104. For example, the longer the duration for which the previously-odorized gas has remained idle in the pipe 104, the longer the fluid evacuation system 102 is operated to evacuate the gas (e.g., the longer the threshold duration). In some examples, in response to the operator determining that the one or more thresholds are satisfied (e.g., block 510 returns a result of YES), control proceeds to block 512. Alternatively, in response to the operator determining that the one or more thresholds are not satisfied (e.g., block 510 returns a result of NO), control returns to block 506.

At block 512, the fluid evacuation system 102 is shut off and/or deactivated. For example, the operator shuts off (e.g., deactivates) the fluid evacuation system 102 so that gas is no longer being evacuated from the pipe 104. In some examples, the fluid evacuation system 102 is removed (e.g., decoupled) from the pipe 104 after deactivation of the fluid evacuation system 102.

At block 514, the gas from the example gas storage 202 is provided to the one or more second example locations 204 of FIG. 2A. For example, the gas storage 202 is fluidly coupled to the second location(s) 204, where the second location(s) 204 correspond to one or more second pipes and/or one or more locations upstream and/or downstream of the pipe 104 of FIGS. 1, 2A, and/or 2B. In some examples, unodorized and/or under odorized gas from the gas storage 202 is provided to the second location(s) 204 to mix with odorized gas at the second location 204. In some examples, the gas is provided to one or more odorizers (e.g., the example odorizer 118 of FIG. 1) prior to entering the second location(s) 204.

While an example manner of implementing the control system circuitry 122 of FIG. 1 is illustrated in FIG. 1, one or more of the elements, processes, and/or devices illustrated in FIG. 1 may be combined, divided, re-arranged, omitted, eliminated, and/or implemented in any other way. Further, the example data interface circuitry 124, the example control activation circuitry 126, and/or, more generally, the example control system circuitry 122 of FIG. 1, may be implemented by hardware alone or by hardware in combination with software and/or firmware. Thus, for example, any of the example data interface circuitry 124, the example control activation circuitry 126, and/or, more generally, the example control system circuitry 122, could be implemented by programmable circuitry in combination with machine readable instructions (e.g., firmware or software), processor circuitry, analog circuit(s), digital circuit(s), logic circuit(s), programmable processor(s), programmable microcontroller(s), graphics processing unit(s) (GPU(s)), digital signal processor(s) (DSP(s)), ASIC(s), programmable logic device(s) (PLD(s)), and/or field programmable logic device(s) (FPLD(s)) such as FPGAs. Further still, the example control system circuitry 122 of FIG. 1 may include one or more elements, processes, and/or devices in addition to, or instead of, those illustrated in FIG. 1, and/or may include more than one of any or all of the illustrated elements, processes and devices.

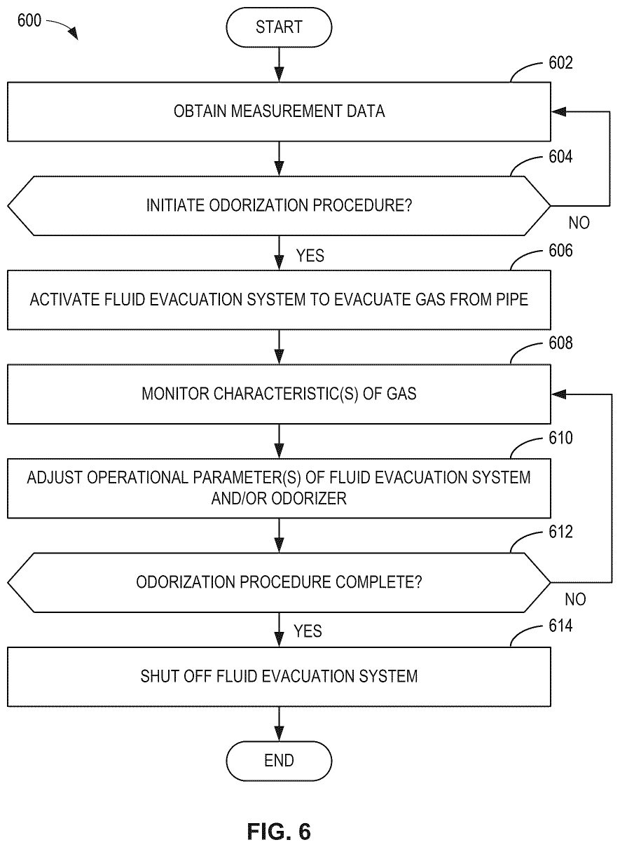

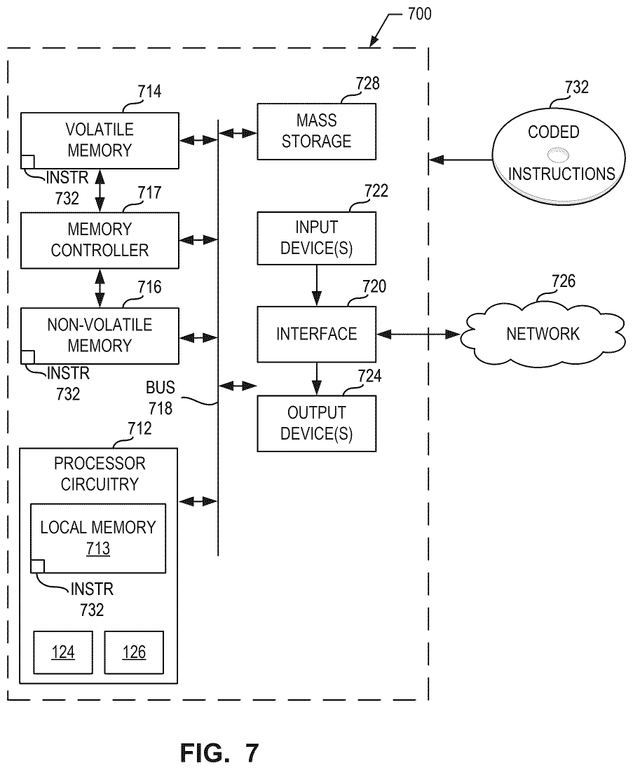

A flowchart representative of example machine readable instructions, which may be executed by programmable circuitry to implement and/or instantiate the control system circuitry 122 of FIG. 6 and/or representative of example operations which may be performed by programmable circuitry to implement and/or instantiate the control system circuitry 122 of FIG. 1, are shown in FIG. 6. The machine readable instructions may be one or more executable programs or portion(s) of one or more executable programs for execution by programmable circuitry such as the programmable circuitry 712 shown in the example processor platform 700 discussed below in connection with FIG. 7. In some examples, the machine readable instructions cause an operation, a task, etc., to be carried out and/or performed in an automated manner in the real world. As used herein, “automated” means without human involvement.

The program may be embodied in instructions (e.g., software and/or firmware) stored on one or more non-transitory computer readable and/or machine readable storage medium such as cache memory, a magnetic-storage device or disk (e.g., a floppy disk, a Hard Disk Drive (HDD), etc.), an optical-storage device or disk (e.g., a Blu-ray disk, a Compact Disk (CD), a Digital Versatile Disk (DVD), etc.), a Redundant Array of Independent Disks (RAID), a register, ROM, a solid-state drive (SSD), SSD memory, non-volatile memory (e.g., electrically erasable programmable read-only memory (EEPROM), flash memory, etc.), volatile memory (e.g., Random Access Memory (RAM) of any type, etc.), and/or any other storage device or storage disk. The instructions of the non-transitory computer readable and/or machine readable medium may program and/or be executed by programmable circuitry located in one or more hardware devices, but the entire program and/or parts thereof could alternatively be executed and/or instantiated by one or more hardware devices other than the programmable circuitry and/or embodied in dedicated hardware. The machine readable instructions may be distributed across multiple hardware devices and/or executed by two or more hardware devices (e.g., a server and a client hardware device). For example, the client hardware device may be implemented by an endpoint client hardware device (e.g., a hardware device associated with a human and/or machine user) or an intermediate client hardware device gateway (e.g., a radio access network (RAN)) that may facilitate communication between a server and an endpoint client hardware device. Similarly, the non-transitory computer readable storage medium may include one or more mediums. Further, although the example program is described with reference to the flowchart(s) illustrated in FIG. 6, many other methods of implementing the example control system circuitry 122 may alternatively be used. For example, the order of execution of the blocks of the flowchart(s) may be changed, and/or some of the blocks described may be changed, eliminated, or combined. Additionally or alternatively, any or all of the blocks of the flow chart may be implemented by one or more hardware circuits (e.g., processor circuitry, discrete and/or integrated analog and/or digital circuitry, an FPGA, an ASIC, a comparator, an operational-amplifier (op-amp), a logic circuit, etc.) structured to perform the corresponding operation without executing software or firmware. The programmable circuitry may be distributed in different network locations and/or local to one or more hardware devices (e.g., a single-core processor (e.g., a single core CPU), a multi-core processor (e.g., a multi-core CPU, an XPU, etc.)). For example, the programmable circuitry may be a CPU and/or an FPGA located in the same package (e.g., the same integrated circuit (IC) package or in two or more separate housings), one or more processors in a single machine, multiple processors distributed across multiple servers of a server rack, multiple processors distributed across one or more server racks, etc., and/or any combination(s) thereof.

The machine readable instructions described herein may be stored in one or more of a compressed format, an encrypted format, a fragmented format, a compiled format, an executable format, a packaged format, etc. Machine readable instructions as described herein may be stored as data (e.g., computer-readable data, machine-readable data, one or more bits (e.g., one or more computer-readable bits, one or more machine-readable bits, etc.), a bitstream (e.g., a computer-readable bitstream, a machine-readable bitstream, etc.), etc.) or a data structure (e.g., as portion(s) of instructions, code, representations of code, etc.) that may be utilized to create, manufacture, and/or produce machine executable instructions. For example, the machine readable instructions may be fragmented and stored on one or more storage devices, disks and/or computing devices (e.g., servers) located at the same or different locations of a network or collection of networks (e.g., in the cloud, in edge devices, etc.). The machine readable instructions may require one or more of installation, modification, adaptation, updating, combining, supplementing, configuring, decryption, decompression, unpacking, distribution, reassignment, compilation, etc., in order to make them directly readable, interpretable, and/or executable by a computing device and/or other machine. For example, the machine readable instructions may be stored in multiple parts, which are individually compressed, encrypted, and/or stored on separate computing devices, wherein the parts when decrypted, decompressed, and/or combined form a set of computer-executable and/or machine executable instructions that implement one or more functions and/or operations that may together form a program such as that described herein.

In another example, the machine readable instructions may be stored in a state in which they may be read by programmable circuitry, but require addition of a library (e.g., a dynamic link library (DLL)), a software development kit (SDK), an application programming interface (API), etc., in order to execute the machine-readable instructions on a particular computing device or other device. In another example, the machine readable instructions may need to be configured (e.g., settings stored, data input, network addresses recorded, etc.) before the machine readable instructions and/or the corresponding program(s) can be executed in whole or in part. Thus, machine readable, computer readable and/or machine readable media, as used herein, may include instructions and/or program(s) regardless of the particular format or state of the machine readable instructions and/or program(s).

The machine readable instructions described herein can be represented by any past, present, or future instruction language, scripting language, programming language, etc. For example, the machine readable instructions may be represented using any of the following languages: C, C++, Java, C #, Perl, Python, JavaScript, HyperText Markup Language (HTML), Structured Query Language (SQL), Swift, etc.

As mentioned above, the example operations of FIG. 6 may be implemented using executable instructions (e.g., computer readable and/or machine readable instructions) stored on one or more non-transitory computer readable and/or machine readable media. As used herein, the terms non-transitory computer readable medium, non-transitory computer readable storage medium, non-transitory machine readable medium, and/or non-transitory machine readable storage medium are expressly defined to include any type of computer readable storage device and/or storage disk and to exclude propagating signals and to exclude transmission media. Examples of such non-transitory computer readable medium, non-transitory computer readable storage medium, non-transitory machine readable medium, and/or non-transitory machine readable storage medium include optical storage devices, magnetic storage devices, an HDD, a flash memory, a read-only memory (ROM), a CD, a DVD, a cache, a RAM of any type, a register, and/or any other storage device or storage disk in which information is stored for any duration (e.g., for extended time periods, permanently, for brief instances, for temporarily buffering, and/or for caching of the information). As used herein, the terms “non-transitory computer readable storage device” and “non-transitory machine readable storage device” are defined to include any physical (mechanical, magnetic and/or electrical) hardware to retain information for a time period, but to exclude propagating signals and to exclude transmission media. Examples of non-transitory computer readable storage devices and/or non-transitory machine readable storage devices include random access memory of any type, read only memory of any type, solid state memory, flash memory, optical discs, magnetic disks, disk drives, and/or redundant array of independent disks (RAID) systems. As used herein, the term “device” refers to physical structure such as mechanical and/or electrical equipment, hardware, and/or circuitry that may or may not be configured by computer readable instructions, machine readable instructions, etc., and/or manufactured to execute computer-readable instructions, machine-readable instructions, etc.

“Including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, it is to be understood that additional elements, terms, etc., may be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, (6) B with C, or (7) A with B and with C. As used herein in the context of describing structures, components, items, objects and/or things, the phrase “at least one of A and B” is intended to refer to implementations including any of (1) at least one A, (2) at least one B, or (3) at least one A and at least one B. Similarly, as used herein in the context of describing structures, components, items, objects and/or things, the phrase “at least one of A or B” is intended to refer to implementations including any of (1) at least one A, (2) at least one B, or (3) at least one A and at least one B. As used herein in the context of describing the performance or execution of processes, instructions, actions, activities and/or steps, the phrase “at least one of A and B” is intended to refer to implementations including any of (1) at least one A, (2) at least one B, or (3) at least one A and at least one B. Similarly, as used herein in the context of describing the performance or execution of processes, instructions, actions, activities and/or steps, the phrase “at least one of A or B” is intended to refer to implementations including any of (1) at least one A, (2) at least one B, or (3) at least one A and at least one B.

As used herein, singular references (e.g., “a”, “an”, “first”, “second”, etc.) do not exclude a plurality. The term “a” or “an” object, as used herein, refers to one or more of that object. The terms “a” (or “an”), “one or more”, and “at least one” are used interchangeably herein. Furthermore, although individually listed, a plurality of means, elements, or actions may be implemented by, e.g., the same entity or object. Additionally, although individual features may be included in different examples or claims, these may possibly be combined, and the inclusion in different examples or claims does not imply that a combination of features is not feasible and/or advantageous.

FIG. 6 is a flowchart representative of example machine readable instructions and/or example operations 600 that may be executed, instantiated, and/or performed by programmable circuitry to implement the control system circuitry 122 of FIG. 1. The example machine-readable instructions and/or the example operations 600 of FIG. 6 begin at block 602, at which the example control system circuitry 122 obtains measurement data associated with gas in the example pipe 104 of FIG. 1. For example, the example data interface circuitry 124 of FIG. 1 obtains and/or accesses the measurement data from the example meter 120 of FIG. 1. In some examples, the measurement data includes at least one of the pressure of the gas in the pipe 104, the volume of the gas, the concentration of odorant relative to the gas in the pipe 104, etc.

At block 604, the example control system circuitry 122 determines whether to initiate an example odorization procedure. For example, the example control activation circuitry 126 of FIG. 1 determines to initiate the example odorization procedure in response to determining, based on the measurement data, that the concentration of odorant in the pipe 104 does not satisfy (e.g., is less than) a threshold concentration. In response to the control activation circuitry 126 determining not to initiate the odorization procedure (e.g., block 604 returns a result of NO), control returns to block 602. Alternatively, in response to the control activation circuitry 126 determining to initiate the odorization procedure (e.g., block 604 returns a result of YES), control proceeds to block 606.

At block 606, the example control system circuitry 122 activates the example fluid evacuation system 102 to evacuate gas (e.g., unodorized and/or under odorized gas) from the pipe 104. For example, the control activation circuitry 126 activates (e.g., turns on) the fluid evacuation system 102 so that the fluid evacuation system 102 can begin compressing and/or evacuating the gas from the pipe 104. In some examples, the fluid evacuation system 102 is coupled to the example odorizer 118 of FIG. 1, the upstream pipe 252 of FIG. 2B, the second location(s) 204 of FIG. 2A, and/or the example gas storage 202 of FIG. 2A to provide the evacuated gas thereto.

At block 608, the example control system circuitry 122 monitors one or more example characteristics of the gas in and/or from the pipe 104. For example, the data interface circuitry 124 monitors the measurement data from the meter 120 to monitor and/or determine a current concentration of odorant in the pipe 104, an amount of gas provided to the odorizer 118 and/or the gas storage 202, a pressure of the gas in the pipe 104, etc. In some examples, the data interface circuitry 124 monitors a duration for which the fluid evacuation system 102 is operating.

At block 610, the example control system circuitry 122 adjusts one or more example operational parameters of the fluid evacuation system 102 and/or the odorizer 118 of FIG. 1. For example, the control activation circuitry 126 can adjust, based on the characteristic(s) of the gas, a compression rate and/or a total compression amount of the fluid evacuation system 102, an injection rate and/or an amount of odorant provided by the odorizer 118, etc.

At block 612, the example control system circuitry 122 determines whether the odorization procedure is complete. For example, the control activation circuitry 126 determines that the odorization procedure is complete in response to determining that the concentration of odorant in the pipe 104 satisfies (e.g., is greater than or equal to) a threshold concentration, the amount (e.g., a volume) of gas provided to the odorizer 118 and/or the gas storage 202 satisfies a threshold amount, a duration for which the fluid evacuation system 102 is operating satisfies a threshold duration, etc. In response to the control activation circuitry 126 determining that the odorization procedure is not complete (e.g., block 612 returns a result of NO), control returns to block 608. Alternatively, in response to the control activation circuitry 126 determining that the odorization procedure is complete (e.g., block 612 returns a result of YES), control proceeds to block 614.

At block 614, the example control system circuitry 122 shuts off (e.g., deactivates) the fluid evacuation system 102. For example, the control activation circuitry 126 shuts off (e.g., deactivates) the fluid evacuation system 102 so that gas is no longer being evacuated from the pipe 104 and/or provided to the odorizer 118. In some examples, the control activation circuitry 126 shuts off the odorizer 118 so that the odorizer 118 no longer injects and/or provides odorant to the gas. In some examples, the example operations 600 of FIG. 6 may be performed in addition to or instead of the example operations 400 of FIG. 4 and/or the example operations 500 of FIG. 5.

FIG. 7 is a block diagram of an example programmable circuitry platform 700 structured to execute and/or instantiate the example machine-readable instructions and/or the example operations of FIG. 6 to implement the control system circuitry 122 of FIG. 1. The programmable circuitry platform 700 can be, for example, a server, a personal computer, a workstation, a self-learning machine (e.g., a neural network), a mobile device (e.g., a cell phone, a smart phone, a tablet such as an iPad™), a personal digital assistant (PDA), an Internet appliance, a DVD player, a CD player, a digital video recorder, a Blu-ray player, a gaming console, a personal video recorder, a set top box, a headset (e.g., an augmented reality (AR) headset, a virtual reality (VR) headset, etc.) or other wearable device, or any other type of computing and/or electronic device.

The programmable circuitry platform 700 of the illustrated example includes programmable circuitry 712. The programmable circuitry 712 of the illustrated example is hardware. For example, the programmable circuitry 712 can be implemented by one or more integrated circuits, logic circuits, FPGAs, microprocessors, CPUs, GPUs, DSPs, and/or microcontrollers from any desired family or manufacturer. The programmable circuitry 712 may be implemented by one or more semiconductor based (e.g., silicon based) devices. In this example, the programmable circuitry 712 implements the example data interface circuitry 124 and the example control activation circuitry 126.

The programmable circuitry 712 of the illustrated example includes a local memory 713 (e.g., a cache, registers, etc.). The programmable circuitry 712 of the illustrated example is in communication with main memory 714, 716, which includes a volatile memory 714 and a non-volatile memory 716, by a bus 718. The volatile memory 714 may be implemented by Synchronous Dynamic Random Access Memory (SDRAM), Dynamic Random Access Memory (DRAM), RAMBUS® Dynamic Random Access Memory (RDRAM®), and/or any other type of RAM device. The non-volatile memory 716 may be implemented by flash memory and/or any other desired type of memory device. Access to the main memory 714, 716 of the illustrated example is controlled by a memory controller 717. In some examples, the memory controller 717 may be implemented by one or more integrated circuits, logic circuits, microcontrollers from any desired family or manufacturer, or any other type of circuitry to manage the flow of data going to and from the main memory 714, 716.

The programmable circuitry platform 700 of the illustrated example also includes interface circuitry 720. The interface circuitry 720 may be implemented by hardware in accordance with any type of interface standard, such as an Ethernet interface, a universal serial bus (USB) interface, a Bluetooth® interface, a near field communication (NFC) interface, a Peripheral Component Interconnect (PCI) interface, and/or a Peripheral Component Interconnect Express (PCIe) interface.

In the illustrated example, one or more input devices 722 are connected to the interface circuitry 720. The input device(s) 722 permit(s) a user (e.g., a human user, a machine user, etc.) to enter data and/or commands into the programmable circuitry 712. The input device(s) 722 can be implemented by, for example, an audio sensor, a microphone, a camera (still or video), a keyboard, a button, a mouse, a touchscreen, a trackpad, a trackball, an isopoint device, and/or a voice recognition system.

One or more output devices 724 are also connected to the interface circuitry 720 of the illustrated example. The output device(s) 724 can be implemented, for example, by display devices (e.g., a light emitting diode (LED), an organic light emitting diode (OLED), a liquid crystal display (LCD), a cathode ray tube (CRT) display, an in-place switching (IPS) display, a touchscreen, etc.), a tactile output device, a printer, and/or speaker. The interface circuitry 720 of the illustrated example, thus, typically includes a graphics driver card, a graphics driver chip, and/or graphics processor circuitry such as a GPU.

The interface circuitry 720 of the illustrated example also includes a communication device such as a transmitter, a receiver, a transceiver, a modem, a residential gateway, a wireless access point, and/or a network interface to facilitate exchange of data with external machines (e.g., computing devices of any kind) by a network 726. The communication can be by, for example, an Ethernet connection, a digital subscriber line (DSL) connection, a telephone line connection, a coaxial cable system, a satellite system, a beyond-line-of-sight wireless system, a line-of-sight wireless system, a cellular telephone system, an optical connection, etc.

The programmable circuitry platform 700 of the illustrated example also includes one or more mass storage discs or devices 728 to store firmware, software, and/or data. Examples of such mass storage discs or devices 728 include magnetic storage devices (e.g., floppy disk, drives, HDDs, etc.), optical storage devices (e.g., Blu-ray disks, CDs, DVDs, etc.), RAID systems, and/or solid-state storage discs or devices such as flash memory devices and/or SSDs.

The machine readable instructions 732, which may be implemented by the machine readable instructions of FIG. 6, may be stored in the mass storage device 728, in the volatile memory 714, in the non-volatile memory 716, and/or on at least one non-transitory computer readable storage medium such as a CD or DVD which may be removable.

From the foregoing, it will be appreciated that example systems, apparatus, articles of manufacture, and methods have been disclosed that re-odorize and/or redistribute unodorized (and/or under odorized) gas from a pipe. The disclosed systems, apparatus, articles of manufacture, and methods implement a fluid evacuation system on the pipe to evacuate and/or compress gas therefrom, then cycle the gas through an example oxidizer before returning the gas to the pipe. Additionally or alternatively, examples disclosed herein utilize the fluid evacuation system to direct the unodorized gas to an example gas storage, from which the unodorized gas can be provided to one or more second locations (e.g., a second pipe and/or a location upstream and/or downstream of the pipe) to mix with odorized gas at the second location(s). Advantageously, by enabling re-odorization and/or redistribution of unodorized gas, the disclosed systems, methods, apparatus, and articles of manufacture reduce risk of environmental harm associated with undetected leakage of gas from a pipe. Additionally, the disclosed systems, methods, apparatus, and articles of manufacture utilize less odorant compared to some known odorizing techniques (e.g., pickling), thus reducing cost of re-odorization and/or reducing likelihood of unnecessary detection of odorant by surrounding residents.

Example gas odorization apparatus, control, and associated methods are disclosed herein. Further examples and combinations thereof include the following:

Example 1 includes an apparatus comprising a pipe to contain a gas, the gas having a first concentration of odorant, and a fluid evacuation system coupled to the pipe and to a second location, the fluid evacuation system to evacuate the gas from the pipe and pump the gas to the second location, the gas at the second location having a second concentration of odorant greater than the first concentration of odorant.

Example 2 includes the apparatus of example 1, wherein the pipe is a first pipe, the second location corresponding to a second pipe upstream of the first pipe.

Example 3 includes the apparatus of example 2, wherein the gas is a first gas, the second pipe to contain a second gas, a combination of the first gas and the second gas corresponding to the second concentration of odorant.

Example 4 includes the apparatus of example 1, wherein the second location corresponds to an odorizer, the odorizer to provide odorant to the gas.

Example 5 includes the apparatus of example 4, wherein the odorizer is fluidly coupled to the pipe, the fluid evacuation system to pump the gas from the odorizer to the pipe.

Example 6 includes the apparatus of example 4, further including a gas storage fluidly coupled to the odorizer, the fluid evacuation system to pump the gas from the odorizer to the gas storage.

Example 7 includes the apparatus of example 1, further including a meter operatively coupled to the pipe, the fluid evacuation system to evacuate the gas when a characteristic of the gas measured by the meter does not satisfy a threshold.

Example 8 includes a method comprising coupling a fluid evacuation system to a pipe, the pipe to contain a gas having a first concentration of odorant, coupling the fluid evacuation system to a second location, and activating the fluid evacuation system to evacuate the gas from the pipe and pump the gas to the second location, the gas at the second location having a second concentration of odorant greater than the first concentration of odorant.

Example 9 includes the method of example 8, wherein the pipe is a first pipe, and wherein coupling the fluid evacuation system to the second location includes coupling the fluid evacuation system to a second pipe upstream of the first pipe.

Example 10 includes the method of example 9, wherein the gas is a first gas, the second pipe to contain a second gas, a combination of the first gas and the second gas corresponding to the second concentration of odorant.

Example 11 includes the method of example 8, wherein coupling the fluid evacuation system to the second location includes coupling the fluid evacuation system to an odorizer, the odorizer to provide odorant to the gas.

Example 12 includes the method of example 11, further including fluidly coupling the odorizer to the pipe, the fluid evacuation system to pump the gas from the odorizer to the pipe.

Example 13 includes the method of example 11, further including fluidly coupling a gas storage to the odorizer, the fluid evacuation system to pump the gas from the odorizer to the gas storage.

Example 14 includes the method of example 8, further including operatively coupling a meter to the pipe, and activating the fluid evacuation system to evacuate the gas when a characteristic of the gas measured by the meter does not satisfy a threshold.

Example 15 includes an apparatus comprising a fluid evacuation system operatively coupled to a pipe to evacuate and compress gas from the pipe, and an odorizer fluidly coupled to the fluid evacuation system, the fluid evacuation system to provide the gas from the pipe to the odorizer and pump the gas from the odorizer to a second location, the odorizer to provide odorant to the gas.

Example 16 includes the apparatus of example 15, further including a meter operatively coupled to the pipe, the meter to measure a characteristic associated with the gas in the pipe.

Example 17 includes the apparatus of example 16, wherein the characteristic includes at least a concentration of odorant relative to the gas in the pipe, a pressure of the gas, or a volume of the gas.

Example 18 includes the apparatus of example 16, further including control circuitry operatively coupled to the fluid evacuation system and to the meter, the control circuitry to obtain the characteristic from the meter, and cause the fluid evacuation system to evacuate the gas in response to a determination that the characteristic does not satisfy a threshold.

Example 19 includes the apparatus of example 15, wherein the pipe is a first pipe, the second location corresponding to a second pipe separate from the first pipe.

Example 20 includes the apparatus of example 15, wherein the second location corresponds to the pipe.

The following claims are hereby incorporated into this Detailed Description by this reference. Although certain example systems, apparatus, articles of manufacture, and methods have been disclosed herein, the scope of coverage of this patent is not limited thereto. On the contrary, this patent covers all systems, apparatus, articles of manufacture, and methods fairly falling within the scope of the claims of this patent.

What is claimed is:

1. An apparatus comprising:
a pipe to contain a gas, the gas having a first concentration of odorant; and
a fluid evacuation system coupled to the pipe and to a second location, the fluid evacuation system to evacuate the gas from the pipe and pump the gas to the second location, the gas at the second location having a second concentration of odorant greater than the first concentration of odorant.

2. The apparatus of claim 1, wherein the pipe is a first pipe, the second location corresponding to a second pipe upstream of the first pipe.

3. The apparatus of claim 2, wherein the gas is a first gas, the second pipe to contain a second gas, a combination of the first gas and the second gas corresponding to the second concentration of odorant.

4. The apparatus of claim 1, wherein the second location corresponds to an odorizer, the odorizer to provide odorant to the gas.

5. The apparatus of claim 4, wherein the odorizer is fluidly coupled to the pipe, the fluid evacuation system to pump the gas from the odorizer to the pipe.

6. The apparatus of claim 4, further including a gas storage fluidly coupled to the odorizer, the fluid evacuation system to pump the gas from the odorizer to the gas storage.

7. The apparatus of claim 1, further including a meter operatively coupled to the pipe, the fluid evacuation system to evacuate the gas when a characteristic of the gas measured by the meter does not satisfy a threshold.

8. A method comprising:
coupling a fluid evacuation system to a pipe, the pipe to contain a gas having a first concentration of odorant;
coupling the fluid evacuation system to a second location; and
activating the fluid evacuation system to evacuate the gas from the pipe and pump the gas to the second location, the gas at the second location having a second concentration of odorant greater than the first concentration of odorant.

9. The method of claim 8, wherein the pipe is a first pipe, and wherein coupling the fluid evacuation system to the second location includes coupling the fluid evacuation system to a second pipe upstream of the first pipe.

10. The method of claim 9, wherein the gas is a first gas, the second pipe to contain a second gas, a combination of the first gas and the second gas corresponding to the second concentration of odorant.

11. The method of claim 8, wherein coupling the fluid evacuation system to the second location includes coupling the fluid evacuation system to an odorizer, the odorizer to provide odorant to the gas.

12. The method of claim 11, further including fluidly coupling the odorizer to the pipe, the fluid evacuation system to pump the gas from the odorizer to the pipe.

13. The method of claim 11, further including fluidly coupling a gas storage to the odorizer, the fluid evacuation system to pump the gas from the odorizer to the gas storage.

14. The method of claim 8, further including operatively coupling a meter to the pipe, and activating the fluid evacuation system to evacuate the gas when a characteristic of the gas measured by the meter does not satisfy a threshold.

15. An apparatus comprising:
a fluid evacuation system operatively coupled to a pipe to evacuate and compress gas from the pipe; and
an odorizer fluidly coupled to the fluid evacuation system, the fluid evacuation system to provide the gas from the pipe to the odorizer and pump the gas from the odorizer to a second location, the odorizer to provide odorant to the gas.

16. The apparatus of claim 15, further including a meter operatively coupled to the pipe, the meter to measure a characteristic associated with the gas in the pipe.

17. The apparatus of claim 16, wherein the characteristic includes at least a concentration of odorant relative to the gas in the pipe, a pressure of the gas, or a volume of the gas.

18. The apparatus of claim 16, further including control circuitry operatively coupled to the fluid evacuation system and to the meter, the control circuitry to:
obtain the characteristic from the meter; and
cause the fluid evacuation system to evacuate the gas in response to a determination that the characteristic does not satisfy a threshold.

19. The apparatus of claim 15, wherein the pipe is a first pipe, the second location corresponding to a second pipe separate from the first pipe.

20. The apparatus of claim 15, wherein the second location corresponds to the pipe.
